(19) United States
(12) Patent Application Publication
(10) Pub. No.: US 2024/0009276 A1
(43) Pub. Date: Jan. 11, 2024

(54) SYNERGISTIC INDUCTION OF IMMUNITY AGAINST RNA VIRUSES

(71) Applicant: Health Research, Inc., Buffalo, NY (US)

(72) Inventor: Pawel KALINSKI, East Aurora, NY (US)

(21) Appl. No.: 18/249,816

(22) PCT Filed: Oct. 22, 2021
(86) PCT No.: PCT/US2021/056330

§ 371 Date: Apr. 20, 2023

Related U.S. Application Data
(60) Provisional application No. 63/105,152, filed on Oct. 23, 2020.
(60) Provisional application No. 63/094,993, filed on Oct. 22, 2020.

(51) Int. Cl. A61K 38/21; A61K 31/713; A61P 37/04; A61P 31/14; A61K 38/19

(52) U.S. Cl. CPC A61K 38/212; A61K 31/713; A61K 38/217; A61P 37/04; A61P 31/14; A61K 38/191

(57) ABSTRACT

Provided are methods for prophylaxis and therapy for viral infections. The methods can facilitate a synergistic anti-viral effect. The method involves administering a combination of agents to an individual in need thereof. The combinations of agents are selected from interferons (IFNs), Toll-Like Receptor (TLR) ligands, polyinosinic:polycytidylic acid, rintatolimod, tumor necrosis factor alpha (TNF-a) or an inducer thereof, and nuclear factor kappa B (NF-xB) or an inducer or activator thereof.

### CROSS-REFERENCE TO RELATED APPLICATIONS

This application claims priority to U.S. provisional patent application No. 63/094,993, filed Oct. 22, 2020, and to U.S. provisional patent application No. 63/105,152, filed Oct. 23, 2020, the disclosures or each of which are incorporated herein by reference.

### BACKGROUND

SARS-Cov-2 is related to SARS, MERS and other coronaviruses, which are positive-sense RNA viruses that generate dsRNA during replication. One of the principal IFN antiviral pathways involves activation of the host RNAses, especially RNase L, which degrades viral RNA, following its activation by OAS1-3¹. Coronaviruses avoid or inhibit upstream pathways that activate type 1 IFNs, including inhibition of signaling by pattern recognition receptors (e.g. RIG-I and MDAS) that sense viral RNA and inhibition of IRF3, a transcriptional factor that induces the expression of type 1 IFNs⁶⁻¹⁰.This inhibition of innate immune responses allows the virus to replicate in host cells, and, in high risk patients, progress to pneumonia and respiratory failure.

In more detail, SARS-CoV-2 and other RNA viruses (which include some of the most common and deadly viruses; such as Influenza, SARS, MERS HIV, HCV, Yellow Fever, Dengue or Ebola viruses), all uses RNA as genetic material. Their ability to suppress, delay or avoid the TLR3- or RIG-I/MDA5-helicase-mediated early activation of IRF3 and induction of the interferon pathway and IFN-responsive genes, allows them to avoiding, delay, and reduce the induction of intracellular defense mechanisms, which involve RNAses (RNA-degrading enzymes) and other intracellular mediators of antiviral immunity such as OAS-1,-2, -3 (OASs are key activators of RNAse-L, involved in degradation of genetic material of RNA viruses'), IFITM3, IFIT1, Mx1, RIG-I, and MDA5), as well as activation of innate immunity, facilitating its spread in the body and its rapid transmission in the susceptible population. Thus, there is an ongoing and unmet need to provide alternative approaches to prophylaxis and therapy for viral infections. The present disclosure is pertinent to this need.

### BRIEF DESCRIPTION OF THE FIGURE

FIG. 1 provides a summary of data showing synergy between double- stranded (ds)RNA species, such as rintatolimod or poly-IC, type-1 and type-2 Interferons (IFNα and IFNγ), and inflammatory cytokine (known NFκB activator) TNFα in the induction of cell-intrinsic mediators of SARS-Co-V2 immunity. Human epithelial cells (SW620) were cultured overnight in the absence or presence of the indicated combinations of rintatolimod (100 ug/ml), IFNα (1000U/ml), IFNγ (1000U/ml) and TNFα (25 ng/ml), before mRNA extraction and Taqman analysis.

### BRIEF SUMMARY

The present disclosure provides approaches for prophylaxis and therapy for viral infections. In embodiments, the disclosure relates to prophylaxis or therapy of an RNA viral infection.

In embodiments, the methods comprise administering a combination of agents to an individual in need thereof. The combination of agents, in various embodiments, prevents or inhibits viral cell entry, prevents or inhibits viral replication, reduces viral load, reduces severity of viral infection, or prevents viral infection. The combinations of agents are selected from interferons (IFNs), Toll-Like Receptor (TLR) ligands, polyinosinic:polycytidylic acid, also referred to in the art as poly I:C, poly(I:C) and PIC, rintatolimod (sold under the tradename _AMPLIGEN), and tumor necrosis factor alpha (TNF-α) or an inducer thereof, and nuclear factor kappa B (NF-κB) or an inducer or activator thereof. In embodiments, the interferon is selected from IFN-α1, IFN-α2, IFN-α8, IFN-α10, IFN-α14, IFN-α21. In embodiments, the IFN-α is any of IFN-α, IFN-β3, IFN-γ, IFN-κ and IFN-ω. In embodiments, the TLR ligands are selected from TLR1, TLR2, TLR3,

TLR4, TLRS, TLR6, TLR7, TLR8, TLR9, TLR10, TLR11, TLR12, and TLR13 ligands. In embodiments, the TLR ligand is a TLR3 ligand. In one embodiment, a TLR3 ligand is a molecule or complex that comprises a pathogen-associated molecular pattern (PAMP). In an embodiment, the TLR3 ligand comprises a single stranded or double stranded RNA. In embodiments, the TLR3 ligand is high molecular weight poly I:C or low molecular weight poly I:C.

The present disclosure relates in part to the observations that IFNα (or closely-related type-1 IFN; IFNβ; known to act through the same receptor) and rintatolimod (or poly-I:C; which also triggers TLR) may show synergy in inducing the genes involved in cellular response to RNA viruses, resulting in intrinsic cell resistance to SARS-CoV-2 and other viruses. The disclosure relates to the observation that a related, but different interferon (IFNy; type-2 IFN, acting through a separate receptor and using only a partially-overlapping signaling pathway) may also show synergy in inducing the genes involved in cellular response to RNA viruses, resulting in intrinsic cell resistance to SARS-CoV-2 and other viruses. The disclosure also includes use of other cell activators (such as TNFa; or TNFa inducers, such as inflammatory cytokines, other TLR-ligands, STING agonists, or inflammasome activators) which may also show synergy with either type-1 or type-2 IFNs; or other IFNs, such as IFNa; IFNI3; IFNy; IFN, in inducing the genes effective against RNA viruses, resulting in intrinsic cell resistance to SARS-CoV-2 and other viruses.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes use of all combinations of agents described herein. Any particular agent or combination of agents may be excluded from the claims.

When reference is made to a Greek character the disclosure includes all forms of representing the Greek character. For example, “α” may be signified by “alpha” or “a” when used.

In embodiments, the disclosure provides for administering combinations of agents that are combinations of at least two of IFNs, TLR ligands, poly(I:C), rintatolimod, and TNF-a). In embodiments, the IFN-a is any of IFN-α, IFN-β, IFN-ϵ, IFN-κ and IFN-ω. In embodiments, the interferon is IFNα that is any of IFN-α1, IFN-α2, IFN-α8, IFN-α10, IFN-α14, IFN-α21. In embodiments, the TLR ligands are selected from TLR1, TLR2, TLR3, TLR4, TLRS, TLR6, TLR7, TLR8, TLR9, TLR10, TLR11, TLR12, and TLR13 ligands. In embodiments, the TLR ligand is a TLR3 ligand. In one embodiment, a TLR3 ligand is a molecule or complex that comprises a pathogen-associated molecular pattern (PAMP). In an embodiment, the TLR3 ligand comprises a single stranded or double stranded RNA. In embodiments, the TLR3 ligand is high molecular weight poly I:C or low molecular weight poly I:C. In embodiments, the TLR3 ligand is rintatolimod. The disclosure includes administering all combinations of the described agents. Any combination of agents administered to an individual can comprise or consist of any two of the described agents. In embodiments, only 2, or only 3, only 4, or only 5 of the described agents are administered and such combination is sufficient to elicit a protective or therapeutic anti-viral effect, either of which may include a synergistic effect. The agents may be administered concurrently or sequentially.

In embodiments, the described methods are used for prophylaxis or treatment of an infection caused by an RNA virus, but the disclosure may also be used for prophylaxis or treatment of DNA viruses. In embodiments, the disclosure pertains to prophylaxis or treatment of infections caused by double stranded and/or single stranded RNA viruses. In non-limiting embodiments, single stranded RNA viruses include any member of the virus family Filoviridae, non-limiting examples of which include hepatitis C virus (HCV), Ebola virus, yellow fever virus, and Dengue virus (DENY). In an embodiment, the described methods are applicable to any infection caused by a member of the virus family Coronaviridae, examples of which include but are not necessarily limited to severe acute respiratory syndrome (SARS), Middle East respiratory syndrome (MERS), feline Coronavirus (FCoV) that can lead to the development of feline infectious peritonitis (FIP), and the virus that causes coronavirus disease 19 (COVID-19/SARS-CoV-2). In embodiments, the disclosure pertains to use of the described combinations of agents for prophylaxis or therapy of infections caused by influenza viruses, including members of the genus Alphainfluenzavirus (e.g., Influenza A virus (IAV), the genus Betainfluenzavirus (e.g., Influenza B virus (IBV), the genus Gammainfluenzavirus (e.g., Influenza C virus (ICV), and the genus Deltainfluenzavirus (e.g. Influenza D virus (IDV). In embodiments, a double stranded RNA virus against which the described methods can be used includes any infectious member of the virus family Birnaviridae, one example of which is Infectious bursal disease virus (IBDV).

In embodiments, the composition is administered to an individual who is at risk for contracting a virus infection or has been infected by a virus. In embodiments, the individual is at risk for contracting an infection, or has an infection, by any of the aforementioned viruses. In a non-limiting embodiment, the individual is at risk of developing or has a Coronavirus infection, including but not necessarily limited to a SARS-CoV-2 infection. In embodiments, the individual is a human and is of an age wherein such risk is heightened, such as any individual over the age of 50 years. In embodiments, the individual has an underlying condition wherein the risk of developing severe symptoms of a Coronavirus infection, such as COVID-19, is increased, including but not necessarily limited to any respiratory condition. In an embodiment, the individual to which a combination of described agents has been diagnosed with COVID-19. In embodiments, the disclosure includes veterinary approaches, such as for administration to domesticated felines who have or are at risk of developing FIP.

In embodiments, a composition of the disclosure is administered to an individual who is infected with SARS-CoV-2, or is suspected of having a SARS-CoV-2 infection, or another RNA virus that causes a deleterious infection. In embodiments, the individual is in need of prophylaxis of treatment of any SARS-CoV-2 variant, including but not necessarily limited to variants currently referred to as variants of interest, variants of concern, and variants of high consequence. In embodiments, the SARS-CoV-2 variants include a spike mutation that is an L452R or E484K spike protein amino acid substitution. In embodiments, the described variant is currently referred to as B.1.1.7, B.1.351, P.1, P.2,

B.1.427, B.1.429, B.1.526.1, or B.1.617.2, the latter currently referred to as the delta variant. In one embodiment, the individual to which a described combination of agents is administered does not have cancer.

In embodiments, an effective amount of a composition is administered to an individual. An effective amount means an amount of a combination of the described agents that will elicit the biological or medical response by a subject that is being sought by a medical doctor or other clinician. In embodiments, an effective amount means an amount sufficient to prevent, or reduce by at least about 30 percent, or by at least 50 percent, or by at least 90 percent, any sign or symptom of viral infection, e.g., any sign or symptom of a viral infection, including but not limited to COVID-19. In embodiments, fever is prevented or is less severe than if the presently described vaccine had not been administered. In embodiments, viral pneumonia is inhibited or prevented. In embodiments, a synergistic anti-viral effect is produced by administering a combination of the described agents.

Administration the described combinations of agents can be performed using any suitable route of administration, including but not limited to parenteral, intraperitoneal, and oral administration. Parenteral infusions include intramuscular, intravenous, intraarterial, intraperitoneal, and subcutaneous administration. The compositions can be administered to humans, and are also suitable for use in a veterinary context and accordingly can be given to non-human animals, including non-human mammals. In embodiments, a single administration is administered and is sufficient for a therapeutic response. In embodiments, more than one administration is provided. Any of the described agents can be obtain from commercial provides, isolated from natural sources, or produced recombinantly, signified by an “r” preceding the particular agent.

Aspects of the present disclosure provide for overcoming previous barriers to effective immunity. Thus, in one embodiment, the disclosure includes use of a combination of rIFNα and Rintatolimod. In previous work, in vitro tests using human coronavirus OC43 and BS-C-1 kidney cells showed an EC50 of 0.4 μg/ml, compared to the easily achievable concentration in humans of 40 μg/ml, (Barnard, D.L., et al. Proc. 16th Intl Conf Antiviral Res, 2003). However, in in vivo studies with SARS-CoV infected mice^{11,12}, early treatment with either Rintatolimod or with IFNa stood out as the only drugs conferring a significant antiviral/survival effect, leading the authors to conclude that Rintatolimod acts via induction of IFNa and thus teaching away from the combined use of both of these agents. In view of key genomic and pathogenic similarities with SARS-CoV, it is expected that embodiments of the disclosure will provide at least partial efficacy against SARS-CoV2, and other viruses as described herein. Based on these observations and known ability of SARS-COV2 and other RNA viruses to block IFNa induction in infected cells, the disclosure demonstrates an unexpected synergy in eliciting antiviral factors, as further described below.

One aspect of the disclosure comprises promoting anti-viral synergy using rintatolimod and poly-I:C, or other IRF3 activators, TLR ligands and other TNFa or TNFa inducers (group 1 factors) or their combinations and any of type-1 or type-2 IFNs; or other IFNs, such as IFNα; IFNβ; IFNγ; IFNγ (group 2 factors) or their combinations, in inducing the genes involved in cellular response to RNA viruses, resulting in intrinsic cell resistance to SARS-CoV2 and other viruses.

Without intending to be constrained by an particular theory, it is considered that because RNA viruses include many common and deadly viruses; such as Influenza, SARS, MERS, HIV, HCV, Yellow Fever, Dengue or Ebola viruses), the current disclosure includes prevention or treatment of viral infections, during seasonal disease outbreaks, pandemics and epidemics, as well as acts of bioterrorism and the treatment of premalignant diseases, such as hepatitis C, and other virally triggered cancers (including DNA viruses; which defense mechanisms partially overlap with RNA viruses).

Thus, in non-limited embodiments, combinations encompassed by this disclosure include but are not necessarily limited to: poly-IC plus IFN-alpha; poly-I:C plus IFN-gamma, AMPLIGEN plus IFN-gamma; TNF-alpha plus IFN-gamma, and TNF-alpha plus IFN-alpha, which are believed to have not been previously described in the present context.

The following Examples are intended to illustrate but not limit the disclosure.

### EXAMPLE 1

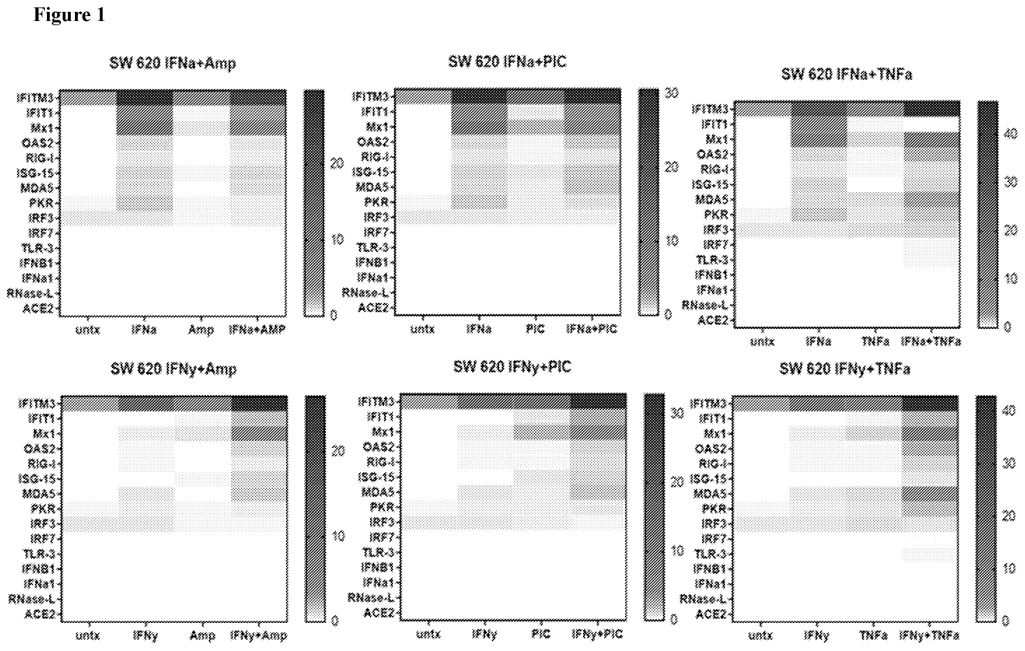

We analyzed cell and tissue samples used to evaluate antiviral synergy using rintatolimod (or poly-I:C) with IFNα, developed as immunostimulatory cocktail with anti-cancer activity in inducing the chemokines mediating intratumoral attraction of CTLs, Th1 and NK cells^{2,3,13}, for the induction of genes involved in SARS-Cov elimination, observing synergistic effects on several of these target genes (see FIG. 1). The synergy in inducing OAS2 (OASs are key activators of RNAse-L, which is not transcriptionally regulated, but activated by OASs which detect dsRNA in order to degrade genetic material of RNA viruses¹), as well as Ifit-1, Mx1, RIG-I, MDA5, TLR3 and several other ISGs/IFN-regulated genes implicated in the intrinsic resistance of epithelial cells to RNA viruses¹, provides support for the application of this combination therapy in patients at early stages of COVID19. A similar synergy in the induction of these cell-intrinsic anti-viral factors was also seen using the combination of TNFα and IFN. The antiviral effects of the AMPLIGEN/IFNα combination against SARS-Cov-2 have been confirmed in BSL3 conditions.

The presented data demonstrate strong synergistic effects between factors which have been previously proposed and or used as individual antiviral agents, thus demonstrating an unexpected advantage of their combined use in antiviral therapy. Further, TLR3 ligands (and other TLR ligands) are considered as inducers of IFNα (which resulted in their past applications as single agents as antiviral factors. Thus, combining IFNα with a TLR3 ligand is has previously not been recommended.

TABLE 1

| | | | | | | |
|---|---|---|---|---|---|---|
| | | Any | Ligands | | | |
| | | TLR3 | for other | | Any TNFα | Any NFκB |
| Poly-IC | rintatolimod | ligand | TLRs | TNFα | Inducer*** | Activator*** |

| | | | | | |
|---|---|---|---|---|---|
| IFNα (any | Yes* | Yes* | Yes* | *** | Yes* |
| species) | | | | | |
| IFNβ (any | Yes** | Yes** | Yes* | *** | Yes** |
| species) | | | | | |
| IFNγ | Yes* | Yes* | Yes* | *** | Yes* |
| IFNλ (any | Yes*** | Yes*** | Yes*** | *** | Yes** |
| species) | | | | | |

*Direct support by current data (Rintatolimod and poly-IC share binding to TLR3 but not other immunostimulatory properties/recognition receptors/signaling pathways³)

The following reference listing is not an indication that any particular reference is material to patentability:

### References:

1. Silverman, R.H. Viral encounters with 2′,5′-oligoadenylate synthetase and RNase L during the interferon antiviral response. J Virol 81, 12720-12729 (2007).
2. Muthuswamy, R., Berk, E., Junecko, B.F., Zeh, H.J., Zureikat, A.H., Normolle, D., Luong, T.M., Reinhart, T.A., Bartlett, D.L. & Kalinski, P. NF-kappaB hyperactivation in tumor tissues allows tumor-selective reprogramming of the chemokine microenvironment to enhance the recruitment of cytolytic T effector cells. Cancer Res 72, 3735-3743 (2012).
3. Theodoraki, M.N., Yemeni, S., Sarkar, S.N., Orr, B., Muthuswamy, R., Voyten, J., Modugno, F., Jiang, W., Grimm, M., Basse, P.H., Bartlett, D.L., Edwards, R.P. & Kalinski, P. Helicase-Driven Activation of NFkappaB-COX2 Pathway Mediates the Immunosuppressive Component of dsRNA-Driven Inflammation in the Human Tumor Microenvironment. Cancer Res 78, 4292-4302 (2018).
4. Muthuswamy, R., Wang, L., Pitteroff, J., Gingrich, J.R. & Kalinski, P. Combination of IFNalpha and poly-I:C reprograms bladder cancer microenvironment for enhanced CTL attraction. J Immunother Cancer 3, 6 (2015).
5. Muthuswamy, R., Corman, J.M., Dahl, K., Chatta, G.S. & Kalinski, P. Functional reprogramming of human prostate cancer to promote local attraction of effector CD8(+) T cells. Prostate 76, 1095-1105 (2016).
6. Chen, X., Yang, X., Zheng, Y., Yang, Y., Xing, Y. & Chen, Z. SARS coronavirus papain-like protease inhibits the type I interferon signaling pathway through interaction with the STING-TRAF3-TBK1 complex. Protein Cell 5, 369-381 (2014).
7. Siu, K.L., Yeung, M.L., Kok, K.H., Yuen, K.S., Kew, C., Lui, P.Y., Chan, C.P., Tse, H., Woo, P.C., Yuen, K.Y. & Jin, D.Y. Middle east respiratory syndrome coronavirus 4a protein is a double-stranded RNA-binding protein that suppresses PACT-induced activation of RIG-I and MDA5 in the innate antiviral response. J Virol 88, 4866-4876 (2014).
8. Yang, Y., Ye, F., Zhu, N., Wang, W., Deng, Y., Zhao, Z. & Tan, W. Middle East respiratory syndrome coronavirus ORF4b protein inhibits type I interferon production through both cytoplasmic and nuclear targets. Sci Rep 5, 17554 (2015).
9. Fang, P., Fang, L., Ren, J., Hong, Y., Liu, X., Zhao, Y., Wang, D., Peng, G. & Xiao, S. Porcine Deltacoronavirus Accessory Protein NS6 Antagonizes Interferon Beta Production by Interfering with the Binding of RIG-I/MDAS to Double-Stranded RNA. J Virol 92(2018).
10. Banerjee, A., Falzarano, D., Rapin, N., Lew, J. & Misra, V. Interferon Regulatory Factor 3-Mediated Signaling Limits Middle-East Respiratory Syndrome (MERS) Coronavirus

Propagation in Cells from an Insectivorous Bat. Viruses 11(2019).
11. Day, C.W., Baric, R., Cai, S.X., Frieman, M., Kumaki, Y., Money, J.D., Smee, D.F. & Barnard, D.L. A new mouse-adapted strain of SARS-CoV as a lethal model for evaluating antiviral agents in vitro and in vivo. Virology 395, 210-222 (2009).
12. Barnard, D.L., Day, C.W., Bailey, K., Heiner, M., Montgomery, R., Lauridsen, L., Chan, P.K. & Sidwell, R.W. Evaluation of immunomodulators, interferons and known in vitro SARS-coV inhibitors for inhibition of SARS-coV replication in BALB/c mice. Antivir Chem Chemother 17, 275-284 (2006).
13. Obermajer, N., Urban, J., Wieckowski, E., Muthuswamy, R., Ravindranathan, R., Bartlett, D.L. & Kalinski, P. Promoting the accumulation of tumor-specific T cells in tumor tissues by dendritic cell vaccines and chemokine-modulating agents. Nat Commun in press(2018).
14. Mailliard, R.B., Alber, S.M., Shen, H., Watkins, S.C., Kirkwood, J.M., Herberman, R.B. &

Kalinski, P. IL-18-induced CD83+CCR7+NK helper cells. J Exp Med 202, 941-953 (2005).
15. Mailliard, R.B., Egawa, S., Cai, Q., Kalinska, A., Bykovskaya, S.N., Lotze, M.T., Kapsenberg, M.L., Storkus, W.J. & Kalinski, P. Complementary dendritic cell-activating function of CD8+and CD4+T cells: helper role of CD8+T cells in the development of T helper type 1 responses. J Exp Med 195, 473-483 (2002).
16. Nakamura, Y., Watchmaker, P., Urban, J., Sheridan, B., Giermasz, A., Nishimura, F., Sasaki, K., Cumberland, R., Muthuswamy, R., Mailliard, R.B., Larregina, A.T., Falo, L.D., Gooding, W., Storkus, W.J., Okada, H., Hendricks, R.L. & Kalinski, P. Helper function of memory

CD8+T cells: heterologous CD8+ T cells support the induction of therapeutic cancer immunity. Cancer research 67, 10012-10018 (2007).
17. Watchmaker, P.B., Urban, J.A., Berk, E., Nakamura, Y., Mailliard, R.B., Watkins, S.C., van

Ham, S.M. & Kalinski, P. Memory CD8+ T cells protect dendritic cells from CTL killing. J Immunol 180, 3857-3865 (2008).
18. Wong, J.L., Mailliard, R.B., Moschos, S.J., Edington, H., Lotze, M.T., Kirkwood, J.M. &

Kalinski, P. Helper activity of natural killer cells during the dendritic cell-mediated induction of melanoma-specific cytotoxic T cells. J Immunother 34, 270-278 (2011).
19. Zaccard, C.R., Watkins, S.C., Kalinski, P., Fecek, R.J., Yates, A.L., Salter, R.D., Ayyavoo, V., Rinaldo, C.R. & Mailliard, R.B. CD40L induces functional tunneling nanotube networks exclusively in dendritic cells programmed by mediators of type 1 immunity. J Immunol 194, 1047-1056 (2015).

1. A method for prophylaxis or therapy of a viral infection, the method comprising administering to an individual in need thereof a combination of at least two of: an interferon (IFN), a Toll-Like Receptor (TLR) ligand, poly(I:C), rintatolimod, tumor necrosis factor alpha (INF-α) or an inducer thereof, nuclear factor kappa β (NF-κB) or an inducer or aedvator thereof

2. The method of claim 1, wherein the TLR ligand is a TLR3 ligand.

3. The method of claim 2, wherein the TLR3 ligand is poly(I:C) or rintatolimod.

4. The method of claim 1, wherein the IFN comprises IFN-α, IFN-β, IFN-γ, IFN-κ or IFN-ω.

5. The method of claim 1, wherein the IFN comprises IFN-α.

6. The method of claim 5, wherein the IFN-α is any of IFN-α1, IFN-α2, IFN-α8, IFN-α10, IFN-α14, or IFN-α21.

7. The method of claim 1, wherein the IFN comprises IFN-γ.

8. The method of claim 1, wherein the combination comprises poly(I:C) and the IFN, wherein the interferon is optionally IFN-α.

9. The method of claim 1, wherein the combination comprises poly(LC) and IFN-α. (Currently amended) The method of claim 1, wherein the combination comprises IFN-α and rintatolimod.

11. The method of claim 1, wherein the combination comprises IFN-α and TNF-α.

12. The method of claim 1, wherein the combination comprises IFN-γ and rintatolimod.

13. The method of claim 1, wherein the combination comprises IFN-γand poly(I:C).

14. The method of claim 1, wherein the combination comprises IFN-γ and TNF-α. (Currently amended) The method of claim 1, wherein the individual is in need of prophylaxis or therapy for an RNA viral infection.

16. The method of claim 15, wherein the combination exhibits a synergistic anti-viral effect.

17. The method of claim 16, wherein the RNA viral infection is due to a virus that is a member of Alphainfluenzavirus, Filoviridae, or Coronaviridae.

18. The method of claim 17, wherein the RNA viral infection is due to a virus that is a Coronaviridae.

19. The method of claim 18, wherein the RNA viral infection is due to a Severe Acute Respiratory Syndrome (SARS) virus.

20. The method of claim 19, wherein h SARS virus is SARS-CoV-2.
